(19) United States
(12) Patent Application Publication
(10) Pub. No.: US 2005/0247311 A1
(43) Pub. Date: Nov. 10, 2005

(54) Reduced-oxygen breathing device

(76) Inventors: Charles Vacchiano, Gulf Breeze, FL (US); G. Merrill Rice, Pensacola, FL (US)

Correspondence Address: Naval Medical Research Center, 503 Robert Grant Avenue, Code OOL, Silver Spring, MD, 20910, US

(21) Appl. No.: 10/959,764

(22) Filed: Oct. 7, 2004

Related U.S. Application Data
(63) Continuation-in-part of application No. 10/244,003, filed on Sep. 16, 2002, now Pat. No. 6,871,645.
(60) Provisional application No. 60/509,091, filed on Oct. 7, 2003.
(60) Provisional application No. 60/591,146, filed on Jul. 27, 2004.

(51) Int. Cl. A61M 15/00; A61M 16/10

(52) U.S. Cl. 128/203.12

(57) ABSTRACT

The Reduced Oxygen Breathing Device (ROBD2) is an apparatus that dilutes the oxygen present in air to concentrations below 21% by mixing the air with nitrogen. The purpose of this dilution is to simulate the reduced oxygen concentration available as one ascends in altitude. The ROBD2 is unique and different from previous devices that reduce the concentration of oxygen in room air via dilution with nitrogen gas in that it uses sophisticated gas regulating devices known as mass flow controllers. The ROBD also employs a gas extraction device as an independent component of the system that can separate nitrogen gas from air for use in the device.

### CROSS-REFERENCE

This application is filed, under 37 CFR 1.53(b), as a continuation-in-part of U.S. application Ser. No. 10/244,003, filed Sep. 16, 2002, herein incorporated by reference. In addition, this application claims priority under 35 USC 119(e) to U.S. Provisional Application No. 60/509,091, filed Oct. 7, 2003 and U.S. Provisional Application No. 60/591,146, filed Jul. 27, 2004, both of which are herein incorporated by reference.

### BACKGROUND OF THE INVENTION

1. Field of the Invention

The present invention relates to a method and apparatus for providing air with a less than ambient concentration of oxygen (reduced-oxygen air) to a human or other subject. More particularly, the invention relates to a method and apparatus for inducing hypoxia in a subject by delivering enriched nitrogen (and, thereby, reduced-oxygen) air to the subject in an isobaric setting to simulate various altitudes above sea level over relatively short periods.

2. Description of Prior Art

Altitude sickness strikes thousands of individuals every year resulting in problems from sleep disorders to pulmonary edemas to death. These individuals are pilots, skiers, mountain climbers, or merely business travelers to high altitude regions. The key to dealing with the altitude sickness is taking advantage of the body's ability to gradually acclimatize through a transition through progressively higher altitudes. Unfortunately, most individuals do not have the time to acclimatize.

The physiology of altitude sickness and the adjustment to altitude is covered in numerous textbooks. An excellent one is “Medicine For Mountaineering” by James Wilkerson, M.D. Copyright 1992, published by The Mountaineers of Seattle, Wash. from which much of the immediately following discussion is derived.

The body adjusts to altitude by increasing respiratory volume, increasing the pulmonary artery pressure, increasing the cardiac output, increasing the number of red blood cells, increasing the oxygen carrying capability of the red blood cells, and even changing body tissues to promote normal function at lower oxygen levels.

For example, at an altitude level of 3,000 feet the body already begins increasing the depth and rate of respiration. As a result of this, more oxygen is delivered to the lungs. In addition, the pulmonary artery pressure is increased which opens up portions of the lung which are normally not used, thus increasing the capacity of the lungs to absorb oxygen. For the first week or so, the cardiac output increases to increase the level of oxygen delivered to the tissues. The body also begins to increase the production of red blood cells. Other changes include the increase of an enzyme (DPG) which, in-turn, facilitates the release of oxygen from the blood and increase the numbers of capillaries within the muscle to better facilitate the exchange of blood with the muscle.

Tissue hypoxia is caused by the body's inability to obtain or utilize an adequate supply of oxygen. Under normal circumstances, there are three main ways by which this can occur. An individual can breathe a gas mixture in which the percentage of oxygen in the inspired air is insufficient to support adequate cellular respiration. This type of hypoxia (hypoxic hypoxia) can be found in situations where gases such as nitrogen or carbon dioxide are present in higher than normal concentrations relative to air at sea level, thereby displacing oxygen in the gas mixture. Breathing a gas mixture that contains approximately the same percentages of gases as found at sea level, but where the total pressure of the gas mixture is reduced causes a second form of hypoxia (hypobaric hypoxia). This is the situation encountered in altitude exposures. Finally, a third form of hypoxia (histiotoxic hypoxia) is caused by certain toxins (e.g. carbon monoxide, cyanide) that interfere with the body's utilization of oxygen at the cellular level.

Physiologically, the response to each of these types of hypoxia is similar as the organism attempts to compensate for the reduced amount of oxygen available for cellular metabolism. The rate and depth of respiration increases and the heart rate also increases. Subjectively, the individual experiences the sensations of shortness of breath and anxiety. If the hypoxia is severe enough, or if compensatory mechanisms cannot be sustained for any reason, other symptoms become apparent. Organs that have a high oxygen demand are affected first. Cognitive processes are impaired, and the subject may experience marked confusion or ataxia. If the hypoxia persists, coma and death result.

Investigators have utilized different mechanisms to study the effects of hypoxia on human physiology. Exposure to hypobaric environments has been the technique most frequently utilized in aviation settings. The military and commercial aviation industry both spend large sums of money annually training aviators to recognize and experience the signs and symptoms of hypoxia. This type of training is accomplished through the use of hypobaric chambers at fixed sites. These chambers have several drawbacks. Because they are expensive to construct and operate, only a limited number of these chambers can be fielded. Despite their relatively large size, however, they are generally too small to incorporate mission simulators into the hypoxic environment. Additionally, any equipment that is placed into the chamber must be extensively tested to ensure that it is compatible with the reduced barometric pressures within the chamber. Some investigators believe that if hypoxia training and flight could be combined, the face validity of the training scenario would be improved, and the overall training benefit would be significantly increased.

Other investigators have utilized mixed-gas hypoxia (i.e., hypoxic hypoxia) for a variety of reasons, most typically to investigate the physiologic effects of breathing gas mixtures containing a reduced percentage of oxygen, and/or an elevated concentration of carbon dioxide. This technique has several drawbacks. Gas mixtures require the ability to accurately blend and compress gases. Premixed gases also require some storage capacity. Typically, several cylinders of gas mixtures are connected in parallel to a manifold, which is in turn connected to the experimental subject. By changing valve settings on the manifold, differing gas mixtures can be administered. Concentrations are, therefore, limited to only those mixtures created before the experiment. Since the gas mixtures are discrete, no intermediate concentrations can be achieved. The gas mixtures can be administered through a conventional breathing apparatus, but the dependence on cylinders of premixed gases outweighs this convenience. However, because these devices also provoke the symptoms of hypoxia, one potentially useful avenue for these devices could be in the simulation of altitude exposure. Experiments have shown that the physical symptoms and performance deficits induced by hypobaric and mixed-gas hypoxia are qualitatively similar.

Certain devices like the present invention have been presented in the literature as being of two fundamental types. The simplest type exhibits a relatively large volume, closed breathing circuit. An experimental subject is connected to the circuit, and breathes off the reservoir, gradually exchanging the gas mixture present in the reservoir with his or her own exhaled gas (re-breathing). Carbon monoxide and water vapor from the subject may or may not be removed from the reservoir, depending on the experimental design. This type of device is limited in several important respects. The rate at which the oxygen in the reservoir is depleted is dependent on the ratio of the subject's minute ventilation volume and the volume of the reservoir. Since this device has no means to replace oxygen in the reservoir, this device cannot maintain a gas mixture at a particular ratio or concentration. The duration of the experiment is therefore limited to the time it takes for oxygen levels in the reservoir to fall to critical levels. Additionally, the concentration of oxygen in the system is constantly changing making interpretation of the results much more challenging.

A more advanced type of re-breathing circuit has been developed that addresses some of the shortcomings of the simple re-breathing loop. In this device, the subject exhales into a mixing loop, and an oxygen sensor monitors the concentration of oxygen in the loop. Computer software compares the actual concentration of oxygen to the expected concentration of oxygen, and oxygen is added to the mixing loop to hold the concentration of oxygen at a preset level. A shortcoming of this system is that carbon dioxide and water vapor must be continuously removed. Volume loss through the absorption of water vapor and carbon dioxide forces the addition of a replacement volume of gas (typically nitrogen) into the circuit. Because this is a re-breathing apparatus, special masks are required for the subject. Masks are connected to the re-breathing loop by two flexible hoses. Because of the weight of the one-way valve system required, and the weight of the hoses, this apparatus is cumbersome to the subject, and is not well suited for operation in small or confined spaces.

Examples of some of these and similar devices are as follows: Gamow (U.S. Pat. No. 5,398,678) discloses a portable chamber to simulate higher altitude conditions by increasing the pressure within the chamber above that of the ambient pressure, whereas the present invention is practiced in isobaric conditions; Lane (U.S. Pat. No. 5,101,819) teaches a method of introducing nitrogen into a flight training hypobaric chamber (not as in the isobaric conditions of the present invention) to simulate the lower oxygen concentrations at higher altitudes for fighter pilots; Kroll (U.S. Pat. No. 5,988,161) teaches a portable re-breathing device using increasing levels of carbon dioxide to displace oxygen and used to acclimate individuals to higher altitudes, whereas the present invention does not employ this use of exhaled gases (re-breathing) to displace the oxygen; Koni, et al. (U.S. Pat. No. 4,345,612) discloses an apparatus for delivery of a regulated flow of anesthetic gases but uses flow rate input data (not direct measurement of the mixed gases as in the present invention) to control release of gases and is not designed to allow for dynamic conditions; Lampotang, et al. (U.S. Pat. No. 6,131,571) also teaches a device for delivery of anesthetic gases but is more concerned with improved mixing of the gases and maintenance of proper pressure (operating as a ventilator) and is fundamentally different from the present invention, again, in both application and operation (pressure differentials, not direct measurement of mixed gases, is the means for computer control and is utilized to maintain proper system volume, not gas concentrations as in the present invention); and, finally, Marshall, et al. (U.S. Pat. No. 6,196,051) teaches an apparatus for determining odor levels in gas streams but utilizes a mass flow sensor at the inlet valve to regulate the flow of gases into the mixing chamber (not by direct measurement of chamber gases as in the present invention).

Each person reacts differently to a loss of oxygen to the brain. Hypoxia, as this condition is termed, can occur at altitudes as low as 8,000 feet, and occurs rapidly at altitudes of 25,000 feet and above. Being able to predict how individuals react to hypoxia is invaluable in preventing aviation fatalities and accidents that occur as a result of lost or impaired consciousness. Employing altitude chambers, military aviation personnel receive periodic hypoxia-familiarization training to mitigate this threat. The Reduced-Oxygen Breathing Device (ROBD) technology was needed to provide an alternative way of determining how an individual will respond under hypoxic conditions, rather than submitting a person to controlled exposure training in an altitude chamber, which has its own drawbacks. Currently, use of an altitude chamber to determine hypoxic response is costly, risky, and inconvenient.

Altitude chambers are expensive, large, and immobile. Getting personnel to them presents expense and logistical problems. Their use occasionally induces DCS or barotraumas, such as ruptured eardrums, sinus problems, headaches, and toothaches. The ROBD on the other hand, is relatively inexpensive, small, and mobile, and can be integrated with flight simulators. The ROBD can be used anywhere in a normal room at ground level to reliably and systematically produce normoxic (sea-level oxygen levels) and hypoxic conditions equivalent to those at altitudes up to 35,000 feet. Tests indicate the hypoxia experience using the ROBD is “essentially the same” as using an altitude chamber. The same subjective symptoms, decrement in cognitive performance, and type of physiological changes are reported by volunteer test subjects. The ROBD presents a cost effective, reliable, safe, mobile alternative to the conventional altitude chamber.

The parent application, U.S. patent application Ser. No. 10/244,003 (herein referred to a the 003′ application, of which the present invention is a continuation-in-part), addressed the shortcomings in the prior art by using a non-rebreathing circuit coupled with computer-controlled gas adjustments. Ambient air is diluted in the 003′ application with nitrogen on a breath-by-breath basis, providing the experimenter with precise control over the inspired concentration of oxygen on an almost instantaneous basis. Carbon dioxide and water vapor exhaled by the subject are released directly into the environment. Absorption is not necessary in the 003′ application. The small size of the 003′ invention makes fitting the device into cramped simulator environments possible, and multiple units may be incorporated into multi-place aircraft simulators. Maintenance of the mixing loop in the 003′ application is low when compared to re-breathing units, since no consumable items are necessary to absorb water vapor and/or carbon dioxide.

The 003′ ROBD is designed to create a selected static or dynamic gas mixture for breathing and is intended to induce a state of hypoxia in the subject. The 003′ reduced-oxygen breathing apparatus is made up of the following minimum elements: a vessel for gas mixing; an ambient air inlet; an outlet to provide the controlled gas mixture to a subject; an oxygen concentration sensor; a nitrogen gas supply; a nitrogen valve; and a controller for gas mixing, whereby the sensor sends a signal to the controller which manipulates said signal and provides an output signal to the nitrogen valve that adjusts the nitrogen gas supply to the gas mixing vessel in accordance with parameters set by an operator.

During the past three years, the Naval Aerospace Medical Research Laboratory (NAMRL) has continued to develop, test, and evaluate the portable open loop of the 003′ ROBD. The 003′ device is capable of reliably delivering sea level equivalent oxygen concentrations of altitudes up to 35,000 ft. A comparison of the subjective and objective signs and symptoms of hypoxia in 70 volunteers showed no significant difference during exposure to altitude in a hypobaric chamber and the ROBD.

The 003′ ROBD consists of an open gas-mixing chamber, which is constructed of schedule 40 polyvinyl chloride (PVC) pipe in the form of a rectangular loop. A quick-disconnect fitting is located on one end of the loop, such that a standard aviator's oxygen mask can be connected as it would be connected in an aircraft. The other end of the apparatus contains a one-way valve that permits entrance of ambient air into the loop during inspiration. An oxygen sensor is mounted in the mixing loop. At the start of inspiration, ambient air is drawn into the loop. A personal computer, executing a NAMRL developed gas mixture control program is used to control and monitor the concentration of oxygen in the loop just downstream from a mixing fan. The measured percentage of oxygen in the loop is compared to a target level of oxygen. If the loop oxygen concentration exceeds the target value, the software controller actuates a solenoid valve connected to a cylinder of nitrogen gas. When the two values match, the solenoid valve is turned off. Conversely, if the concentration of oxygen in the mixing loop is below that of the target value, a solenoid valve connected to an oxygen cylinder is actuated, until again those values match.

Although the 003′ ROBD has been used to generate hypoxia in over 100 hundred volunteers in a research laboratory setting, there is a need to further develop and “harden” the system for transitioning to the fleet and also to the public market. Several safety features and system modifications have been identified as necessary to accomplish this transition to a more suitable ROBD. The word suitable implies a new device that overcomes the deficiencies in the prior art as noted above and that is low cost when mass-produced, portable, durable, reliable, simple to operate and maintain, and has low man-hour and monetary maintenance requirements. In general there are four (4) major functional shortcomings, that when properly implemented, will meet the primary objectives of both the military and commercial markets. Four specific improvements needed to overcome the obstacles noted in the prior art of the 003′ application are as follows:
1. The ROBD control system needs to be converted from a Personal Computer-based (PC) system to an embedded controller-based system.
2. The improved ROBD may be integrated with a nitrogen extractor.
3. The improved ROBD and Gas Extraction system must be “hardened.”
4. Several improved ROBD Operating Characteristics and Parameters.

### SUMMARY OF THE INVENTION

The ROBD2 of the instant invention overcomes the obstacles noted above in the prior art. The ROBD2 is designed to create a programmable gas mixture that can be used for breathing and is intended to induce hypoxia in a test subject. The following is a summary of the major innovations offered by the instant invention:
1. The ROBD2 is a self-contained instrument with integrated keyboard and display. It does not require an external computer.
2. The ROBD2 is microprocessor-controlled with custom software interface to obtain precise blends of nitrogen and air as a function of altitude.
3. The General User Interface has been designed referencing altitude.
4. The ROBD2 uses thermal mass flow controller technology.
5. The ROBD2 may be controlled from the front panel or remotely via RS232.
6. The ROBD2 provides a more accurate blend and a faster response time than the previous technology.
7. The ROBD2 provides positive pressure in the breathing loop for oxygen dump and simulator modes which require positive pressure to simulate certain avionic conditions.
8. A pulse oximeter with display has been integrated into the ROBD2 including an RS232 data stream interface for remote monitoring of the subject under test.
9. User programmable altitude programs to simulate various ascent/descent/hold altitude points.
10. Integrated oxygen sensor for continuous monitoring of the breathing loop. The output of the oxygen sensor is displayed and monitored by the software. Any results outside the expected will automatically shut down the test and activate the oxygen dump.
11. Automatic oxygen sensor calibration to 100% O2 and air.
12. The ROBD2 can automatically adjust to either compressed gas cylinders or nitrogen generator input without impacting the accuracy of the system.
13. The ROBD2 contains automatic self-tests of all major modes.
14. Communication protocol allows for external computer control of the operation.
15. ROBD2 monitors the 100% oxygen source and prevents the systems from running if adequate oxygen is not available.

Accordingly, an object of this invention is to provide a reduced-oxygen breathing device for providing oxygen-reduced (hypoxic)/nitrogen-enriched air (relative to ambient conditions) to a subject.

Another object of the invention is provide a reduced-oxygen breathing device for providing oxygen-reduced/nitrogen-enriched air to a subject and connected to an aircraft flight simulator to provide hypoxia training.

A still further object of the invention is to provide a reduced-oxygen breathing device for providing oxygen-reduced/nitrogen-enriched air to a subject and connected to a treadmill to provide a stress EKG test.

An additional object of this invention is to provide a reduced-oxygen breathing device for providing oxygen-reduced/nitrogen-enriched air to a subject having reduced lung capacity to evaluate the person's fitness for an aircraft flight or travel to a high-altitude location.

A still further object of the invention is to provide a reduced-oxygen breathing device for providing oxygen-reduced/nitrogen enriched air to a subject as a substitute for conventional exercise cardiovascular stress testing. In this model, a patient gradually receives a progressively hypoxic gas mixture, with the intent of increasing cardiac workload while simultaneously reducing the oxygen content of the blood. As the cardiac workload increases, electrocardiographic changes are monitored, as in conventional exercise stress testing. It is anticipated that this methodology will be exceptionally useful in stress testing for patients that are non-ambulatory, or who have orthopedic injuries that preclude the use of conventional exercise testing. It is also felt to be useful in those patients that have contraindications to conventional pharmacologic stress testing.

These and other objects, features and advantages of the present invention are described in or are apparent from the following detailed description of preferred embodiments.

### BRIEF DESCRIPTION OF THE DRAWINGS

The invention will be described with reference to the drawings, in which like elements have been denoted throughout by like reference numerals. The representation in each of the figures is diagrammatic and no attempt is made to indicate actual scales or precise ratios. Any proportional relationships are shown as approximations.

FIG. 1 shows a piping and instrument diagram (P&ID) of one of the preferred embodiments of the ROBD2 and displays an overview of the electrical, pneumatic and electropneumatic components contained within that embodiment.

FIG. 2 shows an example of a front panel layout for one of the preferred embodiments of the ROBD2 and displays the oxygen dump key, the keys for setting various software driven programs, data entry keys, the breathing mask connection, and the pulse oximeter controls.

FIG. 3 shows an example of a rear panel layout for one of the preferred embodiments of the ROBD2 and displays the RS232 port, oxygen sensor meter, breathing loop vent connection, oxygen sensor connection, status output, oxygen/air/nitrogen gas connections, and electrical connection.

FIG. 4 provides a summary of the safety features of one of the preferred embodiments.

FIG. 5 shows pressure changes with altitude.

FIG. 6 shows sea level oxygen equivalents and estimated tidal volumes and respiratory rates at various altitudes.

FIG. 7 shows an alveolar gas table for oxygen concentrations in air at various altitudes and a representative algorithm for calculating the same.

### DETAILED DESCRIPTION OF THE PREFERRED EMBODIMENTS

The present invention is an improvement to the 003′ ROBD which, in part, consisted of the following elements: an open gas-mixing chamber, in the form of a loop; a quick-disconnect fitting located on one end of the loop, allowing fittings such as a standard aviator's oxygen mask; the other end of the apparatus contains a one-way valve for entrance of ambient air into the loop during inspiration; an oxygen sensor is mounted in the mixing loop. In the 003′ ROBD, at the start of inspiration, ambient air is drawn into the loop and a personal computer, executing a NAMRL developed gas mixture control program, was used to control and monitor the concentration of oxygen in the loop just downstream from a mixing fan. The measured percentage of oxygen in the loop of the 003′ invention was compared to a target level of oxygen and operated as follows: if the loop oxygen concentration exceeds the target value, the software controller actuated a solenoid valve connected to a cylinder of nitrogen gas and when the two values match, the solenoid valve is turned off; conversely, if the concentration of oxygen in the mixing loop is below that of the target value, a solenoid valve connected to an oxygen cylinder is actuated, until again those values match.

The instant invention can be thought of as a second generation Reduced Oxygen Breathing Device (ROBD2). ROBD2 is a computerized gas-blending instrument. The system uses Thermal Mass Flow Controllers (MFC) to mix breathing air and nitrogen to produce the sea level equivalent atmospheric oxygen contents for altitudes up to 40,000 feet. The MFCs are calibrated on primary flow standards traceable to the National Institute of Standards and Technology (NIST). NIST is a federal agency whose mission is to develop and promote measurement, standards, and technology to enhance productivity, facilitate trade, and improve the quality of life. Several safety features are built into the ROBD2 to prevent over-pressurization of the Pilot's mask and to prevent reduced oxygen contents below those being requested for a particular altitude. The software is Menu driven. The main operator's menu consists of three selections, simplifying the use of the system for the field operator. Built in self-tests verify all system component functionality before the operation of the system can begin. If any self-tests fail, the system will not operate. The system is designed to work with both bottled gases and gases produced by the gas membrane system.

The present invention, ROBD2, improves on the 003′ ROBD described previously in several ways. The instant invention offers an alternative to the air and nitrogen cylinders with the introduction of an air/nitrogen producing membrane system. The gas-mixing loop of the 003′ ROBD has been replaced in the instant invention by a gas blending system that is based on thermal mass flow controller (MFC) technology. These MFCs have a built in proportional solenoid valve which is controlled via internal electronics. The control of flow in the instant invention is based on the feedback from an internal flow sensor, which uses the thermal conductivity characteristics of gas to determine thermal mass flow. The MFC uses an internal P&ID control loop to achieve consistent, repeatable and stable flow. The strategic layout of plumbing of the instant invention is enough to homogenously mix the gases. This system will produce a gas mixture within 1% of the requested values. The MFC is calibrated on a NIST traceable piston prover primary flow standard, using room air as a source.

The instant invention involves several significant improvements to the 003′ ROBD that can be summarized as follows:
1. The instant ROBD control system has been converted from a personal computer-based (PC) system to an embedded controller-based system.
   The 003′ ROBD design relied on a host computer to function as the primary user interface and control system for device operation. This major improvement to the 003′ ROBD is to convert the control system from an external PC based control to an internal microprocessor based control device. This improvement includes a LCD based display and keypad as the primary user interface. No host computer is required to operate the device. However, the ability to configure and monitor ROBD operation via an external PC is provided in the instant invention. The electronic communication interface between the PC and the ROBD is achieved via Universal Serial Bus (USB)/RS232.
2. The instant ROBD has been integrated with a nitrogen/oxygen gas extractor system.
   This improvement permits the ROBD to function with a nitrogen and oxygen gas extraction system. The integration of the two subsystems is such that the two subsystems function as one system. The establishment of the interface between the two subsystems requires minimal effort or oversight by the user. However, the integration solution does not prevent the use of compressed gas tanks as the source for nitrogen and oxygen and/or for the use of the extractor to provide one gas while a compressed gas source supplies the other gas. Gas extractor controls, monitoring, and calibration devices are imbedded in the extractor component itself. This improvement allows the user to choose between either compressed gas or room air gas extraction of nitrogen and oxygen as supply sources for the improved ROBD.
3. The improved ROBD and Gas Extraction system has been “hardened.”
   The improved ROBD of the instant invention with integrated gas extraction system is “hardened” such that they will continue to function normally after repeated land, sea and air transport. The containers will meet the NEMA 12 standard. The devices will operate after exposure to a three-foot drop shock load at any orientation.
4. Improved ROBD Operating Characteristics.
   The ROBD2 is capable of producing on demand the sea level oxygen equivalent of an altitude range of 0 to 43,000 feet MSL (21 to 2.46% oxygen, Appendix 1). The mask pressure has an operating bandwidth of 0.5 Hz DC with a pressure range of 0 to 20″ of H₂O with a tolerance of 0 to 1.5″ of H₂O and the ability to control the altitude within 200 feet of the commanded value. The preferred methodology to achieve these functional goals and to reduce gas consumption is a control ‘loop’ which regulates the gas flow from the MFCs based on the breathing loop pressure.
5. Internal Pulse Oximeter.
   The improved ROBD of the present application also is capable of monitoring the heart rate and blood oxygen content (oxygen saturation) of the human test subject. This pulse oximeter is integrated into the ROBD2 module and results are displayed on an LCD panel integral to the module.

In brief, the reduced-oxygen breathing apparatus of the instant invention has the following elements:
(a) a thermal mass flow controller for regulating the release of nitrogen gas;
(b) a thermal mass flow controller for regulating the release of ambient air;
(c) a nitrogen gas inlet that is in fluid communication with the nitrogen mass flow controller;
(d) an ambient air inlet that is in fluid communication with the mass flow controller;
(e) an outlet from the nitrogen mass flow controller that is in fluid communication with the nitrogen mass flow controller on one end and providing the controlled release of nitrogen gas to a common hose at the opposite end;
(f) an outlet from the ambient air mass flow controller in fluid communication with the ambient air mass flow controller on one end and providing the controlled release of ambient air to the common hose at the opposite end;
(g) an oxygen concentration sensor that is in fluid communication with said common hose;
(h) a nitrogen gas supply in fluid communication with the nitrogen gas inlet;
(i) an ambient air supply in fluid communication with the ambient air inlet;
(j) a back pressure regulator in fluid communication with the common hose that controls the pressure to the oxygen concentration sensor and pressure differential to the mass flow controllers;
(k) a microprocessor for controlling the releases of the mass flow controllers and thereby regulating the gas component make-up of the gas mixture;
(l) a back-up system for checking the regulation of the gas component make-up of the gas mixture, where the oxygen concentration sensor sends a signal to microprocessor which manipulates the signal and sends an output signal to a display panel that alerts an operator if gas mixture is not within predetermined limits; and
(m) a gas extraction system using molecular sieve technology to deliver nitrogen gas supply and an air compressor in fluid communication with the gas extraction system to deliver the ambient air gas supply.

The Reduced Oxygen Breathing Device 2 (ROBD2 or gas mixer system) is an apparatus that dilutes the oxygen present in air to concentrations below 21% by mixing the air with nitrogen. The purpose of this dilution, as stated above, is to simulate the reduced oxygen concentration available as one ascends in altitude. The ROBD2 is unique and different from previous devices that reduce the concentration of oxygen in room air via dilution with nitrogen gas in that it uses sophisticated gas regulating devices known as Mass Flow Controllers (MFC). A MFC is essentially an electronically controlled valve that regulates flow of a given gas based on the size of the gas molecules (molecular weight) and the temperature of the gas. Each valve is engineered for a specific gas and they are highly accurate and are often used to calibrate other gas delivery devices.

The ROBD2 has 1 MFC for regulation of air flow and 1 MFC for regulation of nitrogen gas flow. The primary components of the ROBD2 gas mixer are: 1) 2 MFCs, as noted above; 2) a microprocessor and associated electronics to control the MFCs and run various software driven simulated altitude scenarios; 3) hoses to direct the gas flow from the MFCs to an external port; 4) an oxygen sensor that monitors the oxygen concentration in the system downstream from the MFCs and used to ensure correct functioning and mixing of air and nitrogen by the MFCs; 5) a pulse oximeter that measures and reports the heart rate and oxygen saturation of the subject breathing on the device and; 6) an emergency system that allows 100% oxygen from an external source (not regulated by a MFC) to be delivered to the breathing port and therefore to the subject. The oxygen supply to the ROBD2 is for emergency purposes only and is not required for the primary dilution and altitude simulation function of the ROBD2. Emergency oxygen is supplied by a compressed gas source.

Air and nitrogen passing through the ROBDs mass flow controllers can be supplied by compressed gas cylinders or by a gas extraction system. The gas extraction device is an independent component of the system and can separate nitrogen gas from air. The gas extraction device contains a compressor that entrains room air from the environment, pressurizes the air and delivers it to a molecular sieve. The molecular sieve separates the air into it primary component parts (oxygen and nitrogen) based on the size of the gas molecules. The nitrogen gas is pumped into a cylindrical container that acts as a reservoir for delivery under constant pressure to the gas mixer. The remaining gas, mostly oxygen, is vented to the environment as a “waste gas.” Some bleed air directly from the compressor is also pumped into a container to supply the gas mixer with the necessary air supply. Both the air and nitrogen containers are fitted with pressure gauges for monitoring the pressure within the containers and to control flow to the gas mixer.

In brief, air and nitrogen either from compressed gas cylinders (tanks) or from the gas extraction component are supplied to the gas mixer via hoses from the source gas to quick disconnect fittings on the back of the gas mixer. Oxygen from a compressed gas cylinder source only is also supplied via a hose to a quick disconnect fitting on the back of the gas mixer solely as an emergency 100% oxygen breathing supply in case of a medical emergency. Once the air and nitrogen enter the gas mixing system, they are routed to their respective MFC. The amount of flow permitted through each MFC controller is determined by the operator who inputs a specific altitude or series of altitude changes into the microprocessor by a keypad and LED interface on the front of the gas mixer. The altitudes inputted are associated with a particular reduced oxygen concentration and the microprocessor software and the electronic control hardware direct the appropriate flow through the air and nitrogen MFC to produce the desired altitudes and their respective oxygen concentrations.

Output from the air and nitrogen MFCs is funneled into a common hose where the oxygen content is double checked by the oxygen sensor noted above and then the gas is routed to a port on the face plate of the gas mixer. A hose with a standard military aviation facemask is connected to this port for delivery of the gas to the test subject.

Example of One Preferred Embodiment of the Instant Invention

The device consists of 2 separate modules that can be linked together or work independently. Module 1, the ‘ROBD2’, is the gas mixture delivery and test control device and consists of the actual gas mixing and delivery device with embedded micro-controller. Module 2 contains the gas extraction system.

Module 1 is capable of independent operation when removed from module 2. Module 1 contains the LCD display, keypad, and RS-232C interface. The embedded micro-controller firmware for module 2 is completely upgradeable. A device driver is provided to allow for configuration and/or monitoring of the module 1 micro-controller using National Instruments, Inc. “LabVIEW” software. A common RS-232C connection allows interface with the micro-controller, the pulse oximeter, and the oxygen analyzer. A dedicated ‘Oxygen Dump’ key/button on Module 1 is provided to immediately override the currently running program and deliver 100% oxygen within the breathing loop within 5 seconds.

Each module fits within a watertight crushproof case. The modules can be an integral part of the transport case or the module can be removed for use. Each module meets NEMA 12 standards when closed.

Both the ROBD2 and the gas extraction system are capable of operating from an input power of either 100 to 240 V/50 to 60 Hz AC. Each module requires a single power cord to supply power to all components of the module.

The gas extractor module is capable of supplying medical grade breathing gases and has external quick disconnect metal connections for attaching the oxygen and nitrogen hoses to the ROBD2 unit.

Each module has been designed to be essentially free of safety hazards that could injure operators, users or maintenance personnel during operation. These safety hazards include but are not limited to sudden, uncontrolled changes in loop pressure, flow rate and reduction in oxygen content below preset value, non-standard wiring or any non-standard electrical or mechanical practice.

The ROBD2 is expandable, that is, control panels can be added or reconfigured, control input devices changed, display devices can be upgraded to higher resolution devices, microprocessor code can be changed/upgraded and uses industry standard components when appropriate. The ROBD is also supportable throughout the systems projected life. Hardware components are generally commercial-off-the-shelf (COTS) products whenever possible to ensure supportability throughout the life cycle. There have been no modifications to any COTS hardware or software that will require special support or will cause incompatibility issues with new releases of the hardware or software product. This allows the ROBD2 to be maintenance friendly with a mean time to replace consumable items such as the oxygen sensor of 5 minutes or less and a repair goal of less than 30 minutes for all replaceable components.

The following are some of the performance characteristics of this preferred embodiment:

General Performance

A. Maximum ascent rate of 1000 ft/second
B. Maximum descent rate of 1000 ft/second
C. Maximum ceiling altitude of 43,000 feet with respect to sea level
D. Minimum ceiling of zero feet (sea level)
E. Operates within ±200 feet of programmed altitude
   ROBD2/Module 1: Gas Mixing and Delivery Module

The design and implementation of the gas mixing subsystem and the mask pressure subsystem is accomplished so that gas usage is minimized over the entire operating altitude range. A dynamic, on-demand, and real time control system approach has been utilized.

A. The ROBD2 has these, among other, capabilities:

1) Performing Basic Hypoxia Recognition Training—Ascend to a ceiling altitude of 43,000 feet and deliver the corresponding sea level oxygen equivalent through that altitude (2.46% oxygen) while maintaining nominal breathing loop pressure (0 to 1.5″ of H₂O).
2) Performing Basic Positive Pressure Breathing Training—Delivery of 21% oxygen (room air equivalent/zero (0) altitude) at 10.5″ of H₂O to the breathing loop.
3) Performing Basic Flight Simulator Hypoxia Training—Ascend to a ceiling altitude of 43,000 feet and deliver the corresponding sea level oxygen equivalent through that altitude while increasing breathing loop pressure (based on the CRU-103/P regulator pressure schedule) commencing at 30,000 feet.
4) Performing Flight Simulator On Board Oxygen System Failure Training—Ascend to a ceiling altitude of 43,000 feet while increasing breathing loop pressure based on the CRU-103/P regulator pressure schedule commencing at 30,000 feet while providing 21% oxygen (room air) to the breathing loop. On command (“hotkey” or RS232 input) the system will produce the oxygen equivalent for the current altitude while maintaining the appropriate mask pressure (if above 30,000 feet).
B. The system contains an oxygen sensor to monitor percent oxygen in the gas supplied to the common gas outlet by the mass flow controllers.
C. While operating in a program mode the operator is able to rapidly make manual altitude changes and/or hold at a desired altitude.
D. The ROBD2 contains a female connection port with a spring-loaded cover for connection to a standard aviators mask (MBU 12P).
E. HOLD, ASCENT and DESCENT rates in the program mode are given in whole minutes only. The potential length of a hold step is 60 minutes, at a minimum.
F. The “GASES MODE” is part of the calibration sequence. A prompt is provided to input the source gas (Cylinder versus Gas Extractor). If cylinder gas is used, the N₂ level is automatically set to 100% N₂. If the Gas Extraction system is used, the system automatically detects the N₂ content and saves this value. If the N₂ content is outside the required limit, the system alerts the operator.
G. The “3%” menu option read “N₂ SOURCE” and displays the N₂ concentration.
H. In the program mode, an “INSERT” step presents a blank line in the step immediately below the cursor.
I. All fields in any existing program step are capable of being edited by the user.
J. New programs contain two (2) “END PROGRAM” steps. The first line of a new program contains a “HOLD AT ZERO ALTITUDE.”
K. Operator generated programs can be saved by name (Alphanumerically).
L. A single serial command is provided to obtain status of all critical parameters of the device. Upon proper issue of the command, the following information can be uploaded from the device to the host PC: date, time, program number or name, pulse rate, SaO₂, command altitude, actual altitude (as derived from the O₂ sensor), percent O₂ (as derived from the O₂ sensor), MSC 1 flow rate, MSC 2 flow rate, and breathing loop pressure.
   Oxygen Analyzer and Sensor
   The oxygen analyzer has the following minimum capabilities:
A. Continuous percent O₂ reading: value is displayed on the LCD panel; has the ability to self calibrate; operating range is 0 to 100% O₂; Resolution: 0.1% O₂; AC powered; sensor requires no maintenance, and be easily and rapidly replaceable by the user; and analyzer data is accessible via the common RS-232 port.
B. Operating Characteristics: temperature range is 10 to 50° C.; relative humidity 5 to 95%, non-condensing; and elevation to 10,000 feet.
C. Storage and Transportation: temperature range −40 to +70° C.; relative humidity 0 to 100%; and elevation to 20,000 feet.
   Pulse Oximetry
   The Pulse Oximeter is integrated into the ROBD2 module and has the following minimal capabilities:
A. Capability to monitor both oxygen saturation and heart rate
B. Oxygen saturation and heart rate is displayed on an LCD panel integral to the ROBD2 module
C. Capability to turn the monitor on/off and set an oxygen saturation and heart rate “low value” with an audible alarm when that value is achieved
D. Function with both finger and ear sensors
E. The SaO₂ and heart rate are updated every one (1) second
F. Oxygen saturation and heart rate data is accessible via the common RS-232 port.
G. Operating Characteristics: temperature 10 to 50° C.; relative humidity 5 to 95%, elevation to 10,000 feet.
H. Accuracy: SaO2: 80 to 100%±2%; 60 to 79%±3%; resolution: ±1%; heart rate: 40 to 235 bpm±1.7%; resolution: 1 bpm.
I. Storage and Transportation: temperature: −40 to 70° C.; relative humidity: 0 to 100%; elevation: to 20,000 feet.
   Hoses
   Hoses used to connect the ROBD to either a gas extraction device or compressed gas cylinders are braided stainless steel, a minimum of 10 feet long, heat resistant, and flexible at a temperature of 15° C. with metal quick-disconnect fittings on both ends. The air, nitrogen and oxygen fittings differ in such a way as to prevent interchanging the gas supply lines to the device. The hoses can be stored within the transport container housing the ROBD2.
   Compressed Gas Regulators
   Dual stage regulators necessary for operating the ROBD2 using compressed medical grade air, nitrogen and oxygen is provided with module 1. The regulators meets the gas delivery pressure requirements of the ROBD2. The regulators fit “H” size compressed gas cylinders and have quick disconnect fittings for use with the hoses described in item 6.5. The regulators are capable of being stored in the transport container housing the ROBD2.
   User Interface and Display
A. Upon start up, this preferred embodiment behaves as follows:
   1) a system warm-up period during which a message is displayed on the LCD screen with a count down timer.
   2) a system self check that verifies the function of the mass flow controllers and the oxygen analyzer by cross checking the amount of oxygen measured in a user-entered simulated altitude.
   2) a system self-check also verifies the correct functioning of the oxygen pressure switch, the oxygen “dump” switch and the pulse oximeter.
   3) the self-check is preceded by automatic calibration of the oxygen sensor at several dilution points and verification of successful calibration via an LCD screen report.
B. The integral LCD screen contains the following displays:
   1) The self-check activity and verification of correct functioning of the oxygen sensor and mass flow controllers is available on an LCD screen report. If the self-check indicates a MFC or oxygen sensor failure, the LCD display directs the operator to the probable area of malfunction.
   2) Oxygen concentration within the breathing loop
   3) Command Altitude
   4) Simulated Altitude
   5) O₂ saturation and pulse rate
   6) Flow rate at which gas is delivered
   7) Pressure in breathing loop
C. A RS-232 port is available for upload of date, time, program number/name, pulse rate, SaO₂, command altitude, actual altitude (derived from the O₂ sensor), % O₂ (derived from the O₂ sensor), MSC 1 flow rate, MSC 2 flow rate, and breathing loop pressure.
E. An embedded digital event timer has capability to start, pause and stop and clock time.
   Nitrogen Generating System
A. System is capable of producing 99% N₂.
B. System can be mated to the ROBD2 or separated from it for transport.
C. Connectors used to connect the gas generating system to the breathing module can interface with compressed gas cylinders as well.
D. Packaged in a similar water proof and shock resistant container as the ROBD
E. On board oxygen analysis for determining oxygen and nitrogen content
F. Operating time keeper
G. Operates on 100 to 240 V/50 to 60 Hz AC
H. Operating and storage and transportation characteristics are equivalent to those required for other components (oxygen analyzer/pulse oximeter).
I. Weight of nitrogen generating system weighs less than 110 pounds per shipping container, 2 containers to house entire system.
J. Sound produced by nitrogen generating system is less than 65 dB at three feet.

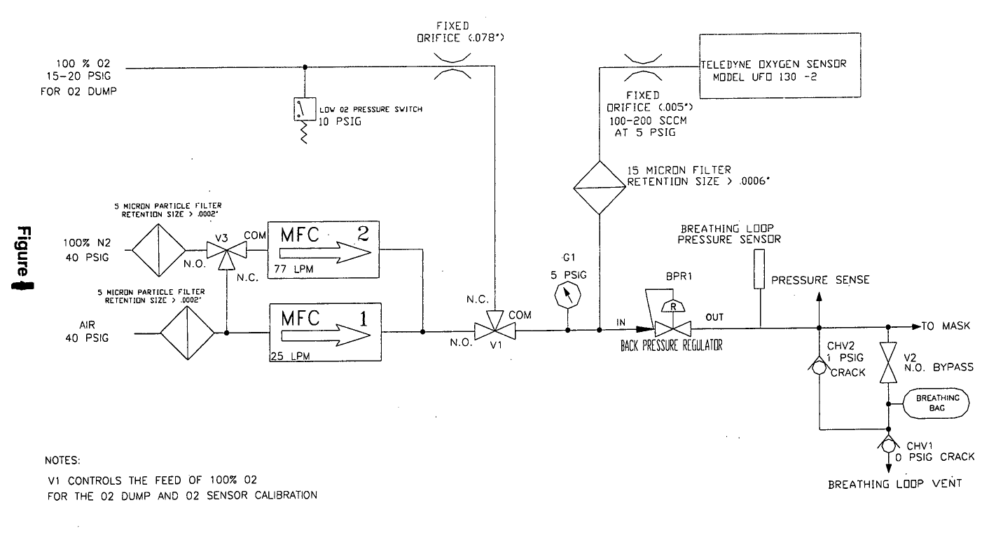

FIG. 1 shows a piping and instrument diagram of one of the preferred embodiments of the ROBD2 and displays an overview of the electrical, pneumatic and electro-pneumatic components contained within that embodiment. There are three gas inputs to the system. Each gas input has a keyed and colored quick connect fitting. The oxygen input is green and requires 15 to 20 PSIG input pressure. The Nitrogen input is blue and requires 40 PSIG input pressure. The Air input is white and requires 40 PSIG input pressure. The input gas lines are ten foot 316 stainless steel flexible braided hoses. The gas enters each respective port and, depending on the programmed altitude, will flow at a specific flow rate through thermal mass flow controllers one (MFC1 for air) and two (MFC2 for nitrogen). The system will produce the correct ratio of air to nitrogen to produce the correct sea level equivalent oxygen content for the programmed altitude. The gas exits each MFC and mixes in the zone between the outputs of the MFCs and the input to back pressure regulator (BPR1). BPR1 serves two purposes. First, the BPR1 controls the pressure to the oxygen sensor's fixed orifice to control the flow at approximately 150 SCCM into the oxygen sensor at all times. The second purpose that BPR1 serves is to control the pressure differential of the MFCs and buffers the MFCs from pressure disturbances of the inhalation and expiratory cycle of the subject under test. All gas connections exiting the BPR1 are considered to be part of the breathing loop. All of the components in the breathing loop are in direct connection with the output port that connects to the pilot's mask. The pressure sense port can be used to connect a mechanical pressure gauge for monitoring breathing loop pressure. This port will normally be plugged when the system is operating. Check valve CHV2 prevents the breathing loop from ever exceeding 1 PSIG (27″ H₂₀). The needle valve adjustment allows each individual system to be setup to produce the positive pressure requirements of the FSHT (Flight Simulator Hypoxia Training), OSFT (Oxygen System Failure Training) and PPT (Positive Pressure Training) modes of the system. Bypass valve V2 closes for positive pressure requirements of the FSHT, OSFT and PPT modes and opens for the HRT (Hypoxia Recognition Training) mode. The requirements of the HRT mode are to keep the breathing loop pressure as close to 0″ as possible. The large orifice bypass valve accomplishes this goal during the HRT mode. The 3-liter breathing bag is externally mounted. This breathing bag satisfies the short, deep quick breaths that supplying a gas mixture with a fixed flow rate from the MFCs cannot satisfy. Check valve CHV1 prevents ambient air from ever being drawn back into the system via the vent port. The vent port will exhaust the gas flow that is not used during the expiratory half of the breathing cycle. Crossover valve V3 allows air to access both flow controllers to satisfy the high flow requirements of the PPT mode and the OSFT mode. Valve V1 controls the flow of 100% oxygen to the pilots mask during an oxygen dump. An oxygen dump is performed when the system operator pressure and emergency dump switch on the front panel. This will normally be done when the operator has determined that the subject under test has become dangerously hypoxic. The mixing action of the MFCs will stop and the output of the MFCs will be isolated from the pilot's mask. The 0.070″ orifice will control the flow of 100% oxygen to the pilots mask. During the oxygen dump, positive pressure aids in getting the gas to the pilots lungs, while the subject under test may not be as capable of taking deep or normal breaths during an induced state of hypoxia. Items related to safety features are the Low 02 pressure switch (10 PSIG) and check valve CHV2.

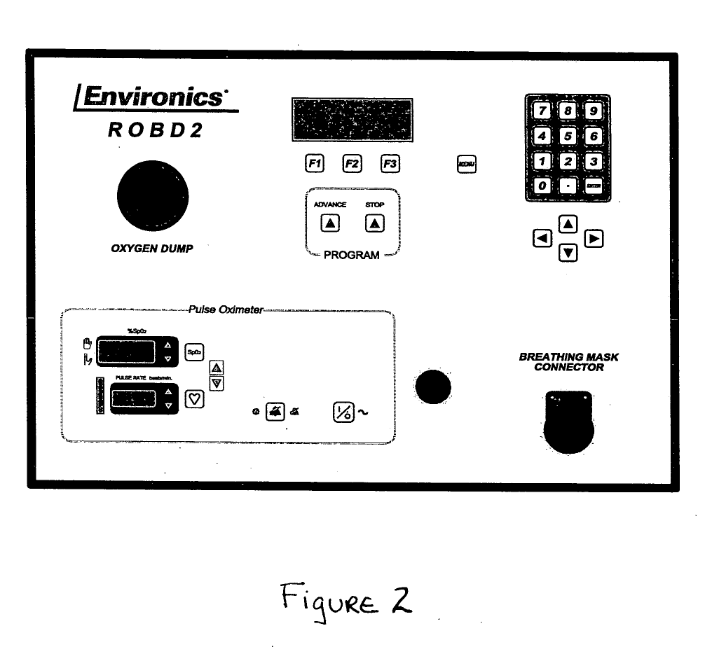

FIG. 2 shows an example of a front panel layout for one of the preferred embodiments of the ROBD2 and displays the oxygen dump key, the keys for setting various software driven programs, data entry keys, the breathing mask connection, and the pulse oximeter controls. The liquid crystal display (LCD) is a four line, 20 characters display, protected by a clear lens. The display is illuminated when the system is in operation. Three function keys (F1, F2 and F3), located below the display, and are used to make various selections from the menu displayed on the bottom line of the screen. The current function of each key is displayed above each function key on the bottom line of the display. The function of each key will change, depending on the current operating mode. The ADVANCE and STOP keys are used while running a program in the Pilot Test Mode (START mode). The STOP key aborts the program immediately upon pressing the key. The ADVANCE key immediately advances the program to the next step upon pressing the key. The numeric keypad is used for data entry of numbers 0 through 9 and a decimal point. Pressing the ENTER key completes the entry of the numeric data selected. The arrow keys are used to move the cursor on the display screen to and from different fields located on the different entry screens or to scroll up or down a menu or list of information. Pressing and holding the arrow keys will cause them to repeat. The MENU key has no function while the system is in the Operator's mode. This key is used to move between multiple menus while the system as in the Administrator (ADMIN) mode. The ADMIN mode is restricted to those who have programming and troubleshooting rights. This emergency stop switch is used to trigger to supply of 100% 02 to the pilot under test. This female connection port (MS 22058-1), with spring-loaded cover, is for the pilot's breathing mask connection. This connector can be used with a finger-tip probe or Y sensor with ear clips.

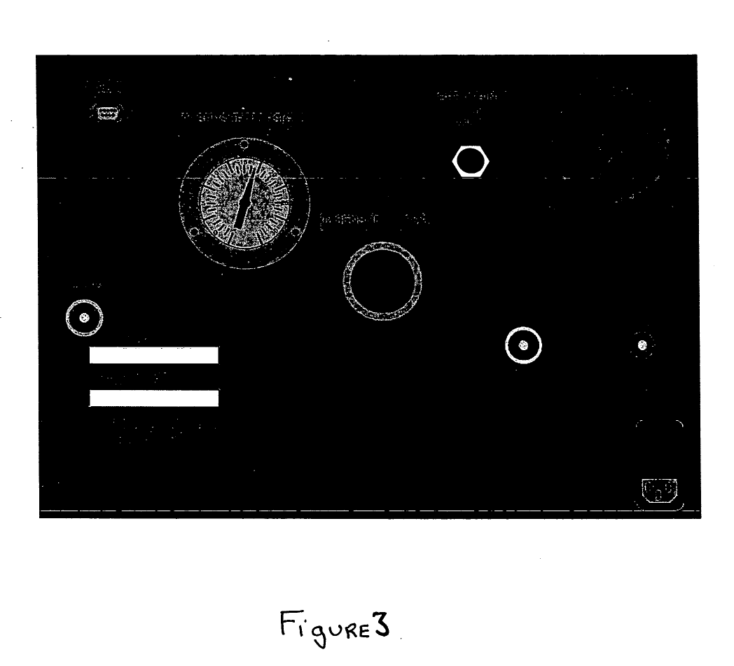

FIG. 3 shows an example of a rear panel layout for one of the preferred embodiments of the ROBD2 and displays the RS232 port, oxygen sensor meter, breathing loop vent connection, oxygen sensor connection, status output, oxygen/air/nitrogen gas connections, and electrical connection. The power entry module supplies AC power to the internal power supplies. The internal power supplies convert and regulate the AC signal to the five DC voltages required by the system electronics. The power entry module has integrated EMI/RFI filtration and switch one or both hot lines dependent upon 110 or 220 VAC operation. The power entry module also has two replaceable fuses. These gas inputs supply source gas to the system components. The quick connect fittings for these ports are colored and keyed. The Nitrogen input is blue, the Air input is white and the oxygen input is green. The Nitrogen and air inputs should be pressurized to a dynamic pressure of 40 PSIG and the oxygen input should be adjusted to a dynamic pressure of 15 to 20 PSIG. One 9-pin RS-232 serial port is connected to the embedded controller of the ROBD system. This port is used for remote control of the ROBD2 using a host computer and communications software. Communication protocol is provided in the programming and technical guide. This protocol can be used to develop control and data collection programs using programs such as National Instruments' Labview. A check valve on this port vents the small amount of excess flow not used during exhalation and also prevents ambient air from being inhaled during inhalation. It also limits the pilot mask pressure. This port is used to connect the latex-free neoprene breathing bag. The breathing bag is used to store mixed gas to satisfy the higher than average inhalation and to satisfy short, quick deep breaths. The cooling fan moves approximately 36 cu/ft per minute of filtered air through the ROBD chassis and out the cooling vents on the top cover of the chassis. The cooling fan should not be obstructed.

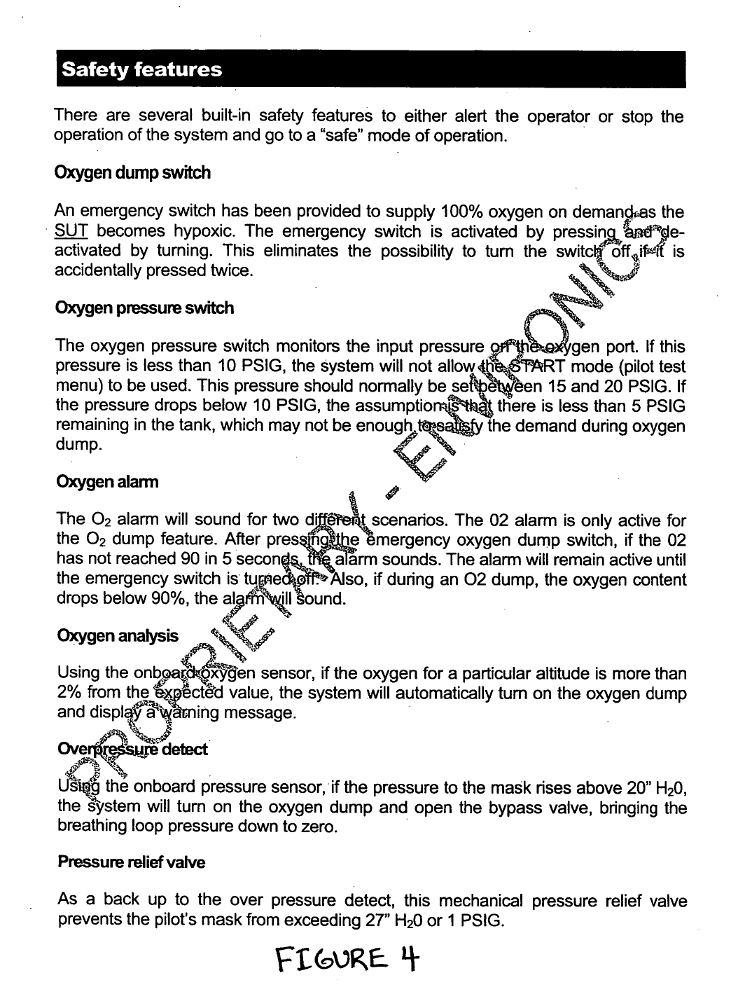

FIG. 4 provides a summary of the safety features of one of the preferred embodiments.

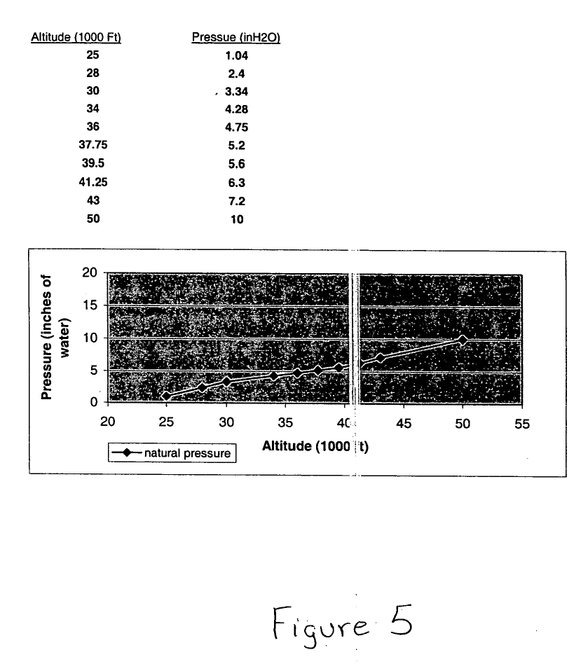

FIG. 5 shows pressure changes with altitude.

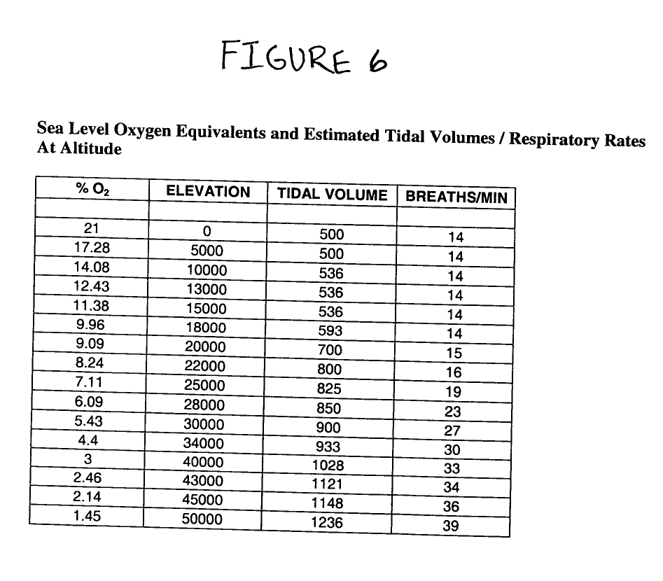

FIG. 6 shows sea level oxygen equivalents and estimated tidal volumes and respiratory rates at various altitudes.

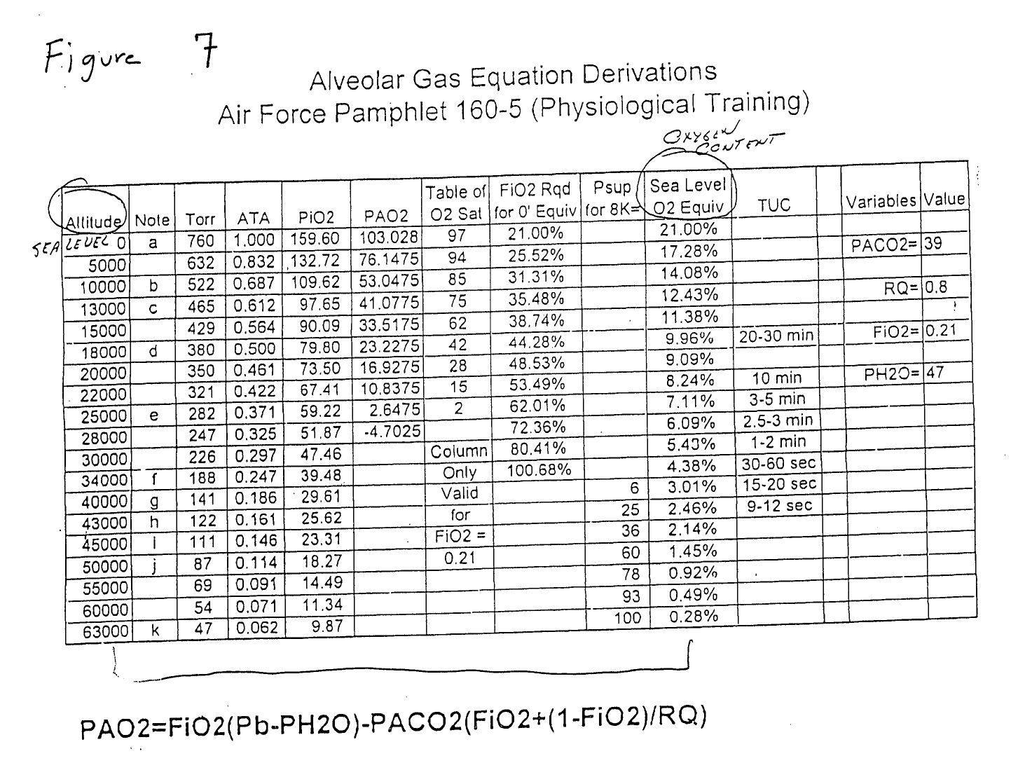

FIG. 7 shows an alveolar gas table for oxygen concentrations in air at various altitudes and a representative algorithm for calculating the same.

The inventors contemplate the following as some of the potential applications for the present invention:
1. For use in conjunction with an aircraft simulator and pilot training
2. For use as a stress EKG test
3. For use in cardio training with a reduced level of exercise required
4. For use in altitude conditioning
5. For use in evaluating a person that has reduced lung capacity and will be exposed to reduced oxygen content
6. For use in providing oxygen-reduced/nitrogen enriched air to a subject as a s substitute for conventional exercise cardiovascular stress testing

Obviously, many modifications and variations of the present invention are possible in light of the above teachings. It is therefore to be understood that, within the scope of the appended claims, the invention may be practiced otherwise than as specifically described.

1. A reduced-oxygen breathing apparatus comprising:
(a) a thermal mass flow controller for regulating the release of nitrogen gas, wherein said nitrogen gas release is for the purpose of contributing to a gas mixture;
(b) a thermal mass flow controller for regulating the release of ambient air, wherein said ambient air release is for the purpose of contributing to said gas mixture;
(c) a nitrogen gas inlet, said inlet being in fluid communication with said nitrogen mass flow controller;
(d) an ambient air inlet, said inlet being in fluid communication with said mass flow controller;
(e) an outlet from said nitrogen mass flow controller, said outlet being in fluid communication with said nitrogen mass flow controller on one end and providing said controlled release of nitrogen gas to a common hose at the opposite end;
(f) an outlet from said ambient air mass flow controller, said outlet being in fluid communication with said ambient air mass flow controller on one end and providing said controlled release of ambient air to said common hose at the opposite end;
(g) a nitrogen gas supply, said nitrogen gas supply being in fluid communication with said nitrogen gas inlet;
(h) an ambient air supply, said ambient air supply being in fluid communication with said ambient air inlet;
(i) a back pressure regulator, said back pressure regulator being in fluid communication with said common hose, wherein said back pressure regulator controls the pressure differential to said mass flow controllers; and
(j) a microprocessor for controlling said releases of said mass flow controllers and thereby regulating the gas component make-up of said gas mixture.

2. The reduced-oxygen breathing device of claim 1, wherein said common hose is in fluid communication with, and is operatively connected to, a delivery unit providing said gas mixture to a subject.

3. The reduced oxygen breathing device of claim 2, wherein said delivery unit is a facemask having:
a one-way valve in fluid communication with said common hose and opening towards said subject, and
a one-way valve opening to the ambient environment for exhalation of said controlled gas mixture by said subject.

4. The reduced oxygen breathing device of claim 3, wherein said facemask is a standard aviator's oxygen mask.

5. The reduced-oxygen breathing device of claim 1, further comprising an oxygen gas supply, said oxygen gas supply being in fluid communication with said common hose.

6. The reduced-oxygen breathing device of claim 5, further comprising an oxygen valve in fluid communication with said common hose and said oxygen gas supply, wherein said oxygen valve is regulated by said microprocessor and controls flow of said oxygen gas supply to said gas common hose.

7. The reduced-oxygen breathing device of claim 1, further comprising an oxygen concentration sensor, said sensor being in fluid communication with said common hose.

8. The reduced-oxygen breathing device of claim 7, further comprising a back-up system for checking said regulation of said gas component make-up of said gas mixture, wherein:
said oxygen concentration sensor sends a signal to said microprocessor;
said microprocessor manipulates said signal;
said microprocessor provides an output signal to a display panel that will alert an operator if said gas mixture is not within predetermined limits set by said microprocessor.

9. The reduced-oxygen breathing device of claim 1, further comprising a gas extraction system using molecular sieve technology to deliver said nitrogen gas supply.

10. The reduced-oxygen breathing device of claim 1, further comprising an air compressor in fluid communication with said gas extraction system to deliver said ambient air gas supply.

11. The reduced-oxygen breathing device of claim 1, further comprising a compressed gas cylinder to deliver said nitrogen gas supply.

12. The reduced-oxygen breathing device of claim 1, further comprising a compressed gas cylinder to deliver said ambient air supply.

13. The reduced-oxygen breathing device of claim 1, further comprising a pulse oximeter in electrical connection with said microprocessor on one end and in physical connection to said subject on the other end.

14. The reduced-oxygen breathing device of claim 1, wherein said physical connection of said pulse oximeter to said subject is at the finger of said subject.

15. The reduced-oxygen breathing device of claim 1, wherein said physical connection of said pulse oximeter to said subject is at the earlobe of said subject.

16. The reduced-oxygen breathing device of claim 1, further comprising an electrical power source connected to said microprocessor, said mass flow controllers, said back pressure regulator, and said oxygen concentration sensor.

17. The reduced-oxygen breathing device of claim 15, wherein data collected by said microprocessor can be accessed via a RS-232 port and uploaded to an external computer.

18. The reduced-oxygen breathing device of claim 1, further comprising an inflatable bladder in fluid communication with said common hose.

19. The reduced-oxygen breathing device of claim 1, further comprising a nitrogen concentration sensor in fluid communication with said common hose.

20. The reduced-oxygen breathing device of claim 1, wherein said microprocessor is programmed to present variable concentrations of oxygen as a function of time.

21. The reduced-oxygen breathing device of claim 20, wherein said variable concentrations of oxygen as a function of time are determined by software of said programmed microprocessor that simulates different test conditions for pilot training.

22. The reduced-oxygen breathing device of claim 9, wherein said gas extraction system has a total weight of less than 220 pounds.

23. The reduced-oxygen breathing device of claim 9, wherein said gas extraction system has a sound level of less than 65 dB measured at three feet.

24. A reduced-oxygen breathing apparatus comprising:
(n) a thermal mass flow controller for regulating the release of nitrogen gas, wherein said nitrogen gas release is for the purpose of contributing to a gas mixture;
(o) a thermal mass flow controller for regulating the release of ambient air, wherein said ambient air release is for the purpose of contributing to said gas mixture;
(p) a nitrogen gas inlet, said inlet being in fluid communication with said nitrogen mass flow controller;
(q) an ambient air inlet, said inlet being in fluid communication with said mass flow controller;
(r) an outlet from said nitrogen mass flow controller, said outlet being in fluid communication with said nitrogen mass flow controller on one end and providing said controlled release of nitrogen gas to a common hose at the opposite end;
(s) an outlet from said ambient air mass flow controller, said outlet being in fluid communication with said ambient air mass flow controller on one end and providing said controlled release of ambient air to said common hose at the opposite end;
(t) an oxygen concentration sensor, said sensor being in fluid communication with said common hose;
(u) a nitrogen gas supply, said nitrogen gas supply being in fluid communication with said nitrogen gas inlet;
(v) an ambient air supply, said ambient air supply being in fluid communication with said ambient air inlet;
(w) a back pressure regulator, said back pressure regulator being in fluid communication with said common hose, wherein said back pressure regulator controls the pressure to said oxygen concentration sensor and pressure differential to said mass flow controllers;
(x) a microprocessor for controlling said releases of said mass flow controllers and thereby regulating the gas component make-up of said gas mixture;
(y) a back-up system for checking said regulation of said gas component make-up of said gas mixture, wherein:
said oxygen concentration sensor sends a signal to said microprocessor;
said microprocessor manipulates said signal;
said microprocessor provides an output signal to a display panel that will alert an operator if said gas mixture is not within predetermined limits set by said microprocessor; and
(z) a gas extraction system using molecular sieve technology to deliver said nitrogen gas supply and an air compressor in fluid communication with said gas extraction system to deliver said ambient air gas supply.

25. A method of inducing hypoxia in a subject in an isobaric environment to simulate various altitudes comprising:
a. fitting said subject with a delivery unit of a reduced-oxygen breathing device wherein the subject can breathe a controlled gas mixture;
b. choosing a concentration of oxygen to be administered via the control means of a microprocessor, wherein a set point is created by an operator, wherein actual and expected oxygen concentrations are compared at a operator-selectable frequency, and wherein adjustments to said controlled gas mixture are made by way of software in said control means to drive mass flow controllers which release nitrogen and ambient air to said controlled gas mixture in said device.
